(19) United States
(12) Patent Application Publication
(10) Pub. No.: US 2013/0144238 A1
(43) Pub. Date: Jun. 6, 2013

(54) ACQUISITION DISTRIBUTION LAYERS PRODUCED FROM CONTINUOUS TOW BANDS AND SYSTEMS AND METHODS RELATING THERETO

(71) Applicants: Sanjay Wahal, Appleton, WI (US); Edward J. Clark, Pearisburg, VA (US)

(72) Inventors: Sanjay Wahal, Appleton, WI (US); Edward J. Clark, Pearisburg, VA (US)

(21) Appl. No.: 13/492,198

(22) Filed: Jun. 8, 2012

Related U.S. Application Data
(63) Continuation-in-part of application No. 13/297,787, filed on Nov. 16, 2011.

(51) Int. Cl. A61F 13/15; D01B 9/00

(52) U.S. Cl. CPC A61F 13/15203; D01B 9/00
USPC 604/367; 19/66 T

(57) ABSTRACT

Generally, an acquisition distribution layer for absorbent hygiene products may be produced from continuous tow bands. A system for producing such an acquisition distribution layer may include, at least, a tow band processing line and a master air jet in operably connected to the tow band processing line to receive a processed tow band from the tow band processing line so as to produce an acquisition distribution layer for an absorbent article.

### CROSS-REFERENCE TO RELATED APPLICATION

This application is a continuation-in-part and claims priority pursuant to 35 U.S.C. §120 to U.S. patent application Ser. No. 13/297,787, filed Nov. 16, 2011, entitled “Nonwoven Materials from Continuous Tow Bands and Apparatuses and Methods Thereof,” which is incorporated by reference herein for all purposes.

### BACKGROUND

The present invention relates to an acquisition distribution layer for absorbent hygiene products produced from continuous tow bands, and to apparatuses, systems, and methods related thereto.

Absorbent hygiene products (“AHP”), such as infant diapers, feminine hygiene pads, adult incontinence products, and the like, have traditionally utilized absorbent structures with various configurations to provide the requisite absorbency performance. Generally, to manage liquid waste, components of an AHP provide for first uptake of a liquid into the absorbent product (typically with a topsheet), then distribution of the liquid within the absorbent product (typically with an acquisition distribution layer), and finally retention of the liquid within the absorbent product (typically in an absorbent core comprising fluff cellulose fibers and superabsorbent polymers). Generally, the various AHP manufacturers use unique topsheets, acquisition distribution layers (“ADL”), and absorbent cores.

An ADL is generally a polyester nonwoven material produced from carding processes with additional filament-to-filament bonding steps to hold the nonwoven material together. Further, to produce ADL materials with complex structures, e.g., a layered structure, the carding processes may require additional steps and apparatuses that can significantly increase cost.

AHP manufacturers typically use third-party manufacturers, which increases cost, to produce the ADL materials due, at least in part to, the high equipment cost and large footprint of carding processes. Further, for a ADL material to integrate into AHP manufacturing lines, third-parties typically provide the ADL materials as large rolls. As a consequence of having to transport the ADL nonwoven materials in the large roll format, a nonwoven material is generally produced with a higher tensile strength so that the integrity and thickness of the nonwoven material is maintained during collection, transportation, and integration into an AHP manufacturing line.

A higher tensile strength is generally achieved during the filament-to-filament bonding steps by using acrylic latex emulsions, ethylene vinyl acetate copolymer latex emulsions, or bicomponent fibers followed by thermal processes to set the filament-to-filament bonding. These extra steps increase expense not only because of the additional steps, but also because acrylic latex emulsions or bicomponent fibers are expensive.

Further, the use of nonwoven materials having higher tensile strength may translate to an ADL with less flexibility, which in an AHP application is not a desirable quality.

Accordingly, systems and methods capable of producing nonwoven materials suitable for use as an ADL that allow for AHP manufacturers to integrate ADL production directly into AHP manufacturing lines would be of benefit to one skilled in the art. Further, systems and methods that provide additional capabilities, like the production of complex structures, without significantly increasing the footprint and/or cost of ADL production would be a further benefit to one skilled in the art.

### SUMMARY OF THE INVENTION

The present invention relates to an acquisition distribution layer for absorbent hygiene products produced from continuous tow bands, and to apparatuses, systems, and methods related thereto.

In one embodiment, the present invention may provide for a system comprising: a tow band processing line; and a master air jet operably connected to the tow band processing line to receive a processed tow band from the tow band processing line so as to produce an acquisition distribution layer for an absorbent article.

In another embodiment, the present invention may provide for a system comprising: a plurality of tow band processing lines; a master air jet in communication with the tow band processing lines to receive a plurality of processed tow bands from the tow band processing lines to form an acquisition distribution layer; and an absorbent hygiene product manufacturing line in communication with the master air jet for receiving the acquisition distribution layer.

In yet another embodiment, the present invention may provide for a method comprising: producing an acquisition distribution layer from a plurality of processed tow bands; and forming an absorbent hygiene product that includes the produced acquisition distribution layer.

In another embodiment, the present invention may provide for a method comprising: processing a plurality of tow bands along at least one tow band processing line to form a plurality of processed tow bands; and combining the plurality of processed tow bands using a master air jet to form an acquisition distribution layer for an absorbent article.

In one embodiment, the present invention may provide for a method comprising: forming a plurality of processed tow bands along a plurality of tow band processing lines; combining in a master air jet the plurality of processed tow bands to form an acquisition distribution layer; adding the acquisition distribution layer to an absorbent hygiene product manufacturing line; and producing an absorbent hygiene product comprising the acquisition distribution layer.

In another embodiment, the present invention may provide for an acquisition distribution layer comprising a plurality of continuous filaments.

In yet another embodiment, the present invention may provide for an absorbent hygiene product comprising, in order: a topsheet; an acquisition distribution layer comprising a plurality of continuous filaments; an absorbent core; and a backsheet.

In another embodiment, the present invention may provide for an absorbent hygiene product comprising: a topsheet; a backsheet; an acquisition distribution layer disposed about an absorbent core; and wherein the acquisition distribution layer is disposed between the topsheet and the backsheet.

The features and advantages of the present invention will be readily apparent to those skilled in the art upon a reading of the description of the preferred embodiments that follows.

### BRIEF DESCRIPTION OF THE DRAWINGS

The following figures are included to illustrate certain aspects of the present invention and should not be viewed as exclusive embodiments. The subject matter disclosed is capable of considerable modifications, alterations, combinations, and equivalents in form and function, as will occur to those skilled in the art and having the benefit of this disclosure.

FIGS. 1A-C illustrate nonlimiting examples of systems according to the present invention for producing an acquisition distribution layer from tow bands suitable for use as described herein.

FIG. 2 illustrates nonlimiting examples of cross-sectional shapes and compositions of bicomponent fibers.

FIG. 3 illustrates nonlimiting examples of the composition of an acquisition distribution layer that can be achieved from processed tow band configurations using systems according to the present invention.

FIG. 4 illustrates a perspective view of a nonlimiting example of a master air jet of the present invention for use in conjunction with a system of the present invention.

FIG. 5 illustrates a side view, partially in section, of a nonlimiting example of a master air jet of the present invention for use in conjunction with a system of the present invention.

FIG. 6 illustrates a top view of the housing of a nonlimiting example of a master air jet of the present invention for use in conjunction with a system of the present invention.

FIG. 7 illustrates an end view illustrating the outlet opening in the housing of a nonlimiting example of a master air jet of the present invention for use in conjunction with a system of the present invention.

FIGS. 8A-B illustrate a view of two different embodiments of the side plates of the housing of a nonlimiting example of a master air jet of the present invention for use in conjunction with a system of the present invention.

FIG. 9 illustrates an end view of the inlet opening of the housing of a nonlimiting example of a master air jet of the present invention for use in conjunction with a system of the present invention.

FIG. 10 illustrates a perspective view of a nonlimiting example of a master air jet of the present invention for use in conjunction with a system of the present invention.

FIG. 11 illustrates a view of one of the side plates of the housing of a nonlimiting example of a master air jet of the present invention for use in conjunction with a system of the present invention.

FIG. 12 illustrates a perspective view of a nonlimiting example of a master air jet of the present invention for use in conjunction with a system of the present invention.

FIGS. 13A-D are pictures at various stages of processing a tow band along a nonlimiting embodiment of a system according to the present invention.

FIGS. 14A-B are pictures at various stages of processing a tow band along a nonlimiting embodiment of a system according to the present invention.

### DETAILED DESCRIPTION

The present invention relates to an acquisition distribution layer for absorbent hygiene products produced from continuous tow bands, and to apparatuses, systems, and methods related thereto.

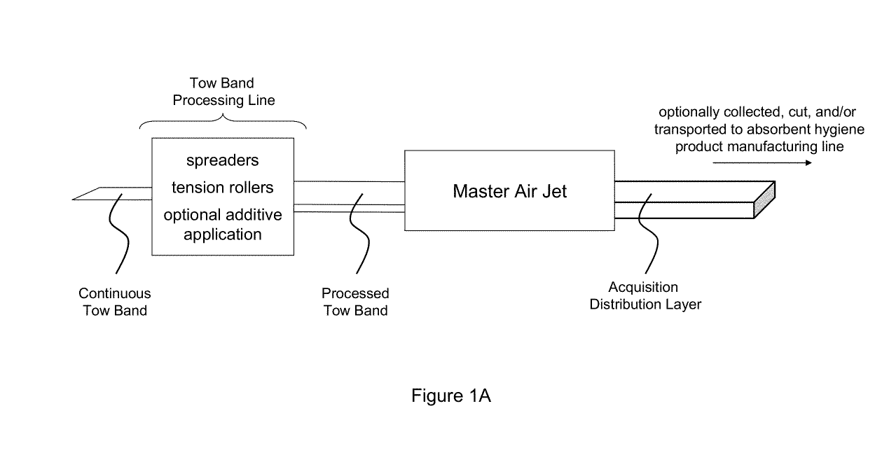

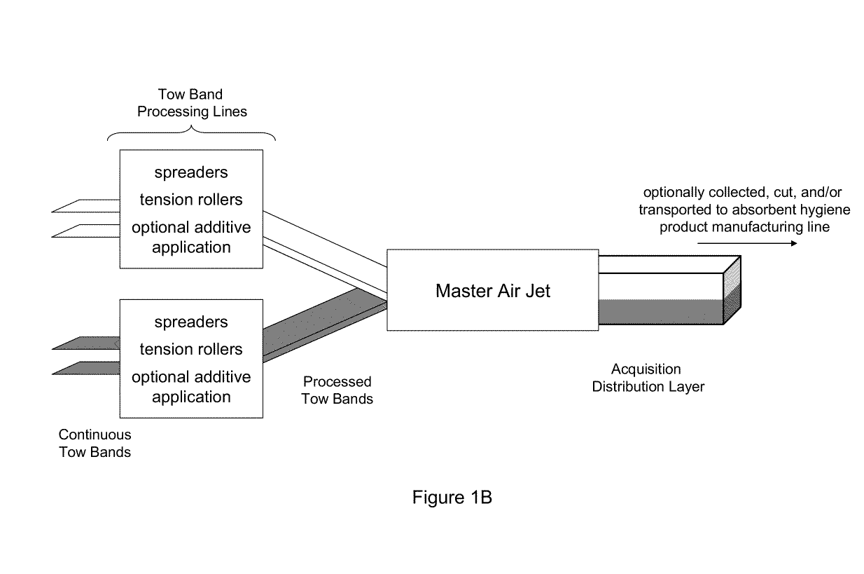

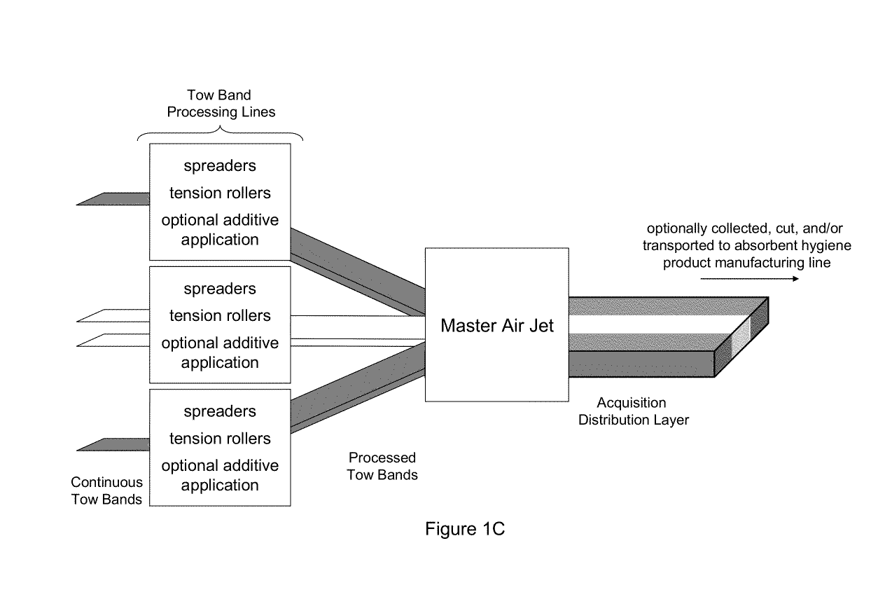

The systems described herein enable the production of an acquisition distribution layer (“ADL”) from a continuous tow band. As illustrated in the nonlimiting examples of FIGS. 1A-C discussed below, in at least some embodiments, a system of the present invention comprises at least one tow band processing line and at least one master air jet. These systems have a much smaller footprint relative to current ADL production lines, i.e., carding manufacturing lines, perhaps as much as 50 times smaller. The much smaller footprint may, among other factors, enable a system of the present invention to be integrated into the production lines of absorbent hygiene products (“AHP”). Because in-line production of an ADL would eliminate many outsourcing, transportation, and handling processes described above, an ADL produced in line with an AHP manufacturing line may require less tensile strength, thereby enhancing flexibility and consequently comfort to the customer. Further, in-line production of an ADL using a system of the present invention may, in some embodiments, advantageously provide AHP manufacturers an avenue to enhance the performance of an ADL, while reducing the cost of the AHP overall. For example, a system of the present invention may be capable of producing, in some embodiments, a higher loft and lower density ADL, which may have enhanced comfort for the consumer, while presenting cost savings to the manufacturer.

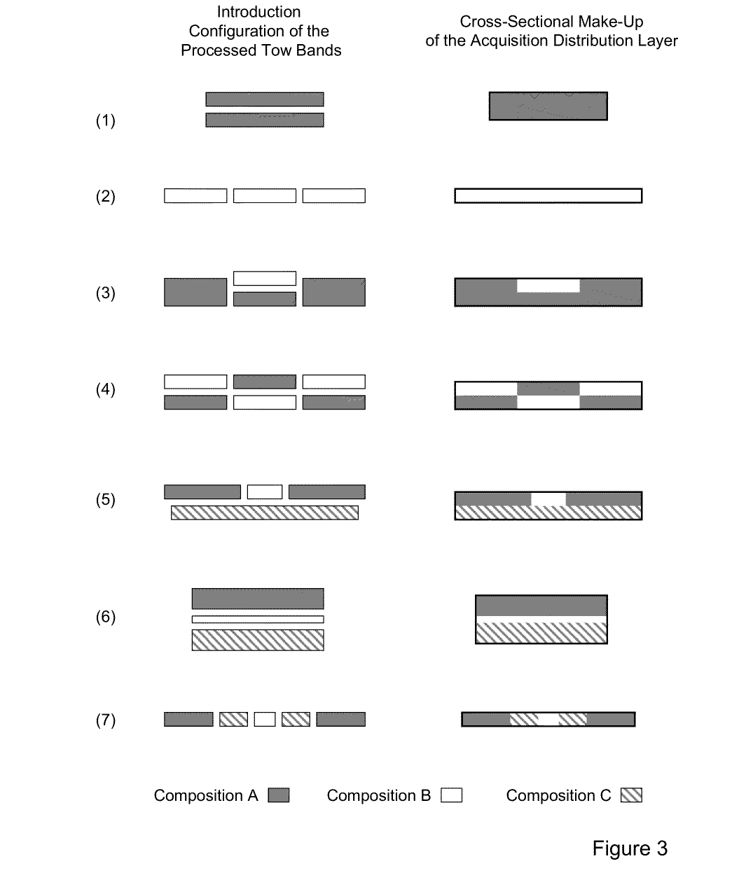

A system of the present invention may, in some embodiments, allow for straightforward production of an ADL with a desired structure and/or composition, which may yield improvements in the performance of an AHP. For example, a system of the present invention may, in some embodiments, allow for the incorporation of additives into an ADL that enhance absorbency and/or reduce odor in an AHP. Additionally, a system of the present invention may, in some embodiments, enable the production of an ADL with a unique structure, as described further in relation to FIG. 3, which may enhance the distribution of fluids to an absorbent core of an AHP.

It should be noted that when “about” is provided below in reference to a number in a numerical list, the term “about” modifies each number of the numerical list. It should be noted that in some numerical listings of ranges, some lower limits listed may be greater than some upper limits listed. One skilled in the art will recognize that the selected subset will require the selection of an upper limit in excess of the selected lower limit.

### I. Acquisition Distribution Layer

Generally, an ADL of the present invention may comprise continuous filaments as opposed to staple fibers used in traditional carding processes described above. The use of continuous filaments and a system that includes a master air jet may advantageously, in some embodiments, provide for an ADL having the necessary integrity without the use of expensive acrylic latex emulsions or bicomponent fibers to enhance filament-to-filament bonding.

In some embodiments of the present invention, an ADL may comprise more than one type of filament as characterized by, inter alia, composition, cross-sectional shape, denier per filament, any other suitable characteristic, or any combination thereof. Production of an ADL comprising more than one type of filament may be as a result of using more than one tow band and/or using at least one tow band having more than one type of filament. A continuous tow band suitable for use in conjunction with the present invention may, in some embodiments, comprise more than one type of filament as characterized by, inter alia, composition, cross-sectional shape, denier per filament, any other suitable characteristic, or any combination thereof. In some embodiments, tow band processing lines for use in conjunction with the present invention may process more than one type of tow bands. In some embodiments, tow band processing lines for use in conjunction with the present invention may process more than one tow band as characterized by, inter alia, filament composition, filament cross-sectional shape, denier per filament, total denier, any other suitable characteristic, or any combination thereof.

Examples of suitable continuous tow bands for use in conjunction with the present invention may include, but not be limited to, continuous tow bands that include carbon filaments, activated carbon filaments, natural fibers, synthetic filaments, bicomponent fibers, or any combination thereof. Suitable continuous tow bands for use in conjunction with the present invention may also comprise filaments that comprise polyolefins, polyethylenes, polypropylenes, polyesters, polyethylene terphalate (PET), lyocells (e.g., TENCEL®, a lyocell, available from The Lenzing Group), viscose, rayons, polyamines, polyamides, polypropylene oxides, polyethylene sulfides, polyphenylene sulfide, liquid crystalline polymeric substances capable of being formed into fibers, silks, wools, cottons, rayons, polyacrylates, polymethacrylates, cellulose acetates, cellulose diacetates, cellulose triacetates, cellulose propionates, cellulose butyrates, cellulose acetate-propionates, cellulose acetate-butyrates, cellulose propionate-butyrates, starch acetates, acrylonitriles, vinyl chlorides, vinyl esters, vinyl ethers, and the like, any derivative thereof, any blend polymer thereof, any copolymer thereof, or any combination thereof.

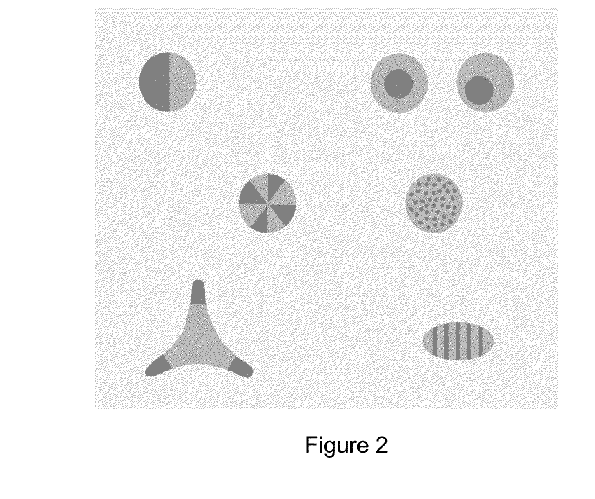

While the systems and methods of the present invention may not require a bicomponent fiber for, inter alia, bonding processes, bicomponent fibers may be used in conjunction with the present invention for bonding processes or to enhance other properties of an ADL. Suitable configurations for bicomponent fibers for use in conjunction with the present invention may include, but not be limited to, side-by-side, sheath-core, segmented-pie, islands-in-the-sea, tipped, segmented-ribbon, or any hybrid thereof, nonlimiting examples of which are illustrated in FIG. 2.

In some embodiments, a continuous tow band, or at least some filaments thereof, may comprise additives. Additives may be integral to the filaments of a continuous tow band (e.g., included in the production of the filaments) or applied to the surface of filaments of a continuous tow band. Suitable additives are detailed further herein and additive application methods are detailed above.

Filaments of a continuous tow band may, in some embodiments, have any suitable cross-sectional shape, including, but not limited to, circular, substantially circular, crenulated, ovular, substantially ovular, polygonal, substantially polygonal, dog-bone, “Y,” “X,” “K,” “C,” multi-lobe, and any hybrid thereof. As used herein, the term “multi-lobe” refers to a cross-sectional shape having a point (not necessarily in the center of the cross-section) from which at least two lobes extend (not necessarily evenly spaced or evenly sized).

In some embodiments, continuous tow bands for use in conjunction with the present invention may have a denier per filament (dpf) ranging from a lower limit of about 1, 2, 3, 5, 10, 12, 15, or 16 to an upper limit of about 50, 40, 30, 20, 15, 12, 10, 7, or 5, and wherein the denier per filament may range from any lower limit to any upper limit and encompass any subset therebetween. In some embodiments, continuous tow bands may have a denier per filament of about 50 or less, and most preferably 10 or less.

While a system of the present invention may be capable of processing a continuous tow band having a total denier of about 10,000 to about 3,000,000, a continuous tow band for use in conjunction with the production of an ADL with a system of the present invention may preferably process a tow band having a total denier ranging from about 25,000 to about 150,000, or more preferably about 25,000 to about 100,000, or most preferably about 25,000 to about 50,000, including any subset therebetween. By way of nonlimiting example, a continuous tow band for use in conjunction with the present invention may comprise cellulose acetate filaments and have a total denier of 100,000 or less and a dpf of about 10 or less. By way of another nonlimiting example, a continuous tow band for use in conjunction with the present invention may comprise polyester filaments and have a total denier of about 60,000 or less and a dpf of about 10 or less.

While a system of the present invention may be capable of processing a continuous tow band with a width of about 60 cm or less, a continuous tow band for use in conjunction with the production of an ADL with a system of the present invention may preferably process a tow band having a width of about 1 cm to about 10 cm, or more preferably about 1 cm to about 5 cm, including any subset therebetween.

While a system of the present invention may be capable of processing a processed tow band having a caliper of about 0.1 mm to about 100 mm, a process tow band for use in conjunction with the production of an ADL with the system of the present invention may preferably process the process tow band having a caliper of about 0.1 mm to about 5 mm, or more preferably about 0.1 mm to about 2 mm, including any subset therebetween.

In some embodiments, a system of the present invention may be configured to produce an ADL from tow bands such that the ADL has a caliper and/or complex cross-sectional make-up not previously realized.

Some embodiments of the present invention may involve combining two or more processed tow bands using a master air jet to produce an ADL. Said processed tow bands may be the same or different. In some embodiments of the present invention, the plurality of tow band processing lines may produce the same processed tow bands. In some embodiments of the present invention, the plurality of tow band processing lines may produce more than one type of processed tow band as characterized by, inter alia, composition, cross-sectional shape of the filaments, dpf of the filaments, total denier of the continuous tow bands, caliper of the processed tow bands, bulk density of the processed tow bands, any other suitable characteristic, or any combination thereof.

In some embodiments of the present invention, the master air jet may be configured to receive the processed tow bands side-by-side with minimal to no overlap, stacked with substantial overlap, or any combination thereof. In some embodiments, the master air jet may be configured to receive a plurality of processed tow bands to produce an ADL with a cross-sectional make-up that substantially resembles the compositional and positional relationship of the plurality of processed tow bands introduced into the master air jet, for example as shown in FIG. 3. It should be noted that in some embodiments in passing through the master air jet of the present invention, the processed tow band compositions are expected to entangle at their interfaces, and therefore the ADL will have a larger degree of entanglement and a cross-sectional make-up substantially resembling the compositional and positional relationship of the processed tow bands as introduced. The degree to which the cross-sectional make-up of the ADL resembles the compositional and positional relationship of the tow bands as introduced will depend on, inter alia, the configuration and processing parameters of the master air jet and the size and shape of the processed tow bands introduced, e.g., higher caliper processed tow bands introduced in a stacked configuration will yield an ADL with better defined layers than would lower caliper processed tow bands.

An ADL of the present invention may, in some embodiments, have a variety of cross-sectional make-ups based on the configuration in which processed tow bands are introduced into master air jet. FIG. 3 illustrates a variety of ADL cross-sectional make-ups with possible corresponding processed tow band introduction configurations. From top to bottom, FIG. 3 illustrates (1) two equally sized processed tow bands of composition A in a stacked configuration yielding an ADL approximately of composition A twice the caliper and substantially the same width; (2) three equally sized processed tow bands of composition B in a side-by-side configuration yielding an ADL of composition B substantially the same caliper and approximately three times the width of an individual processed tow band; (3) two equally sized processed tow bands of composition A and composition B in a stacked configuration that are in a side-by-side configuration between two processed tow bands of composition A of approximately twice the caliper as the internal tow bands yielding an ADL approximately substantially the same caliper as the outer tow bands having a center stripe on one side of composition A with the remainder being composition B; (4) six equally sized processed tow bands configured in two rows of three, the top row being compositions B-A-B and the bottom row being A-B-A yielding an ADL with a checkerboard cross-sectional make-up, a caliper approximately twice that of a single bulked to band from which it was produced, and a width approximately three times that of a single bulked tow band from which it was produced; (5) four processed tow bands in a two row configuration with the top row being three tow bands having substantially the same caliper of compositions A-B-A in a side-by-side configuration where the processed tow bands of composition A are just over twice the width of the processed tow band of composition B and with the bottom row being a single processed tow band of composition C having a caliper approximately that of the top row tow bands and a width approximately that of the total width of the top row yielding an ADL having one side of a single composition (composition C) and the other side being composition A with a small width strip of composition B down the center; (6) three processed tow bands having substantially the same width in a stacked configuration of compositions A-B-C where processed tow bands of composition A and C are substantially the same caliper with approximately three times the caliper of processed tow band of composition B yielding an ADL having a sandwiched configuration with one side being composition A, the other side being composition C, and the middle being a thin (small caliper) of composition B; and (7) five processed tow bands of substantially the same caliper in a side-by-side configuration of compositions A-C-B-C-A where processed tow bands of composition C are slightly wider (approximately 1.3 times) than the processed tow band of composition B and processed tow bands of composition A are approximately twice the width of the processed tow band of composition B yielding an ADL of approximately the same caliper as the individual processed tow bands having a stripped composition A-C-B-C-A with the stripes being approximately the width of the corresponding processed tow bands. It should be noted, that while FIG. 3 may show clear demarcations in the ADL between different compositions, one skilled in the art, with the benefit of this disclosure, should understand that the interface between compositions will be a mixture of the compositions. The degree of mixing at the interface may depend, inter alia, on the setting of the master air jet (e.g., air flow and plate separation), speed at which the processed tow bands are processed through the master air jet, and the caliper and composition of the processed tow bands.

In some embodiments, an ADL of the present invention may have a caliper ranging from about the height of to about 20% greater than the height of the outlet of the master air jet. While a system of the present invention may be capable of producing an ADL or ADL-like material having a caliper ranging from about 0.1 mm to about 100 mm, an ADL of the present invention may preferably have a caliper of about 0.1 mm to about 10 mm, or more preferably about 0.1 mm to about 2 mm, including any subset therebetween. By way of nonlimiting example, an ADL of the present invention having a caliper of about 5 mm may be utilized in a diaper. By way of another nonlimiting example, an ADL of the present invention having a caliper of about 1 mm may be utilized in a feminine hygiene product.

In some embodiments, an ADL of the present invention may have a bulk density of about 0.05 g/cm³ or less. In some embodiments, an ADL of the present invention may have a bulk density ranging from a lower limit of about 0.005 g/cm³ or 0.01 g/cm³ to an upper limit of about 0.1 g/cm³, 0.05 g/cm³, or 0.01 g/cm³, and wherein the bulk density of an ADL may range from any lower limit to any upper limit and encompass any subset therebetween.

In some embodiments, an ADL of the present invention may have a width substantially the same as the width of the widest processed tow band(s) from which it is formed. In some embodiments, an ADL of the present invention may have a width substantially the same as the sum of the width of processed tow bands from which it is formed. While a system of the present invention may be capable of producing an ADL or ADL-like material having a width ranging from about 5 cm to about 10 m, an ADL of the present invention may preferably have a width of about 2 cm to about 10 cm, or more preferably about 4 cm to about 8 cm, including any subset therebetween. By way of nonlimiting example, a polyester tow having 55,000 total denier and an initial width of about 2 cm may be run through a tow band processing line having three spreaders and a delivery roll and then introduced to a master air jet at width of about 10 cm to produce an ADL having a caliper of about 1 cm and a width of about 10 cm.

### II. Systems for the Production of an Acquisition Distribution Layer and Methods Relating Thereto

In some embodiments, a system of the present invention for producing an ADL from a tow band may comprise at least one tow band processing line operably connected to at least one master air jet so as to receive a processed tow band therefrom, nonlimiting examples of which are illustrated in FIGS. 1A-1C. Generally, a tow band processing line converts a tow band into a processed tow band, and a master air jet utilizing a Venturi flow converts a processed tow band into an acquisition distribution layer.

In some embodiments, tow band processing lines may include apparatuses and/or machinery to spread tow bands, uncrimp tow bands, open tow bands, bulk tow bands, apply additives to tow bands, or any combination thereof. One skilled in the art should understand the apparatuses and/or machinery needed to spread tow bands, uncrimp tow bands, bulk tow bands, apply additives to tow bands, or any combination thereof. Nonlimiting examples of suitable apparatuses and/or machinery may include spreaders, tension rollers, guide rollers, air bulking jets, sprayers, steamers, and the like, or any combination thereof. For example, a rod maker from HAUNI may be modified so as to provide the spreaders and tension rollers for opening the tow bands prior to introduction into a master air jet. Examples of air bulking jets are described in more detail herein and can be found in U.S. Pat. Nos. 6,253,431 and 6,543,106, the entire disclosures of which are incorporated herein by reference.

A system of the present invention may, in some embodiments, include at least one tow band processing line operably connected to a master air jet. A master air jet generally uses an air jet to create a Venturi that moves processed tow bands through the master air jet apparatus. The Venturi may further act to entangle filaments of adjacent processed tow bands as they pass through the master air jet. In some embodiments, the master air jet of the present invention may be configured to received a plurality of processed tow bands.

Referring now to a nonlimiting example illustrated in FIG. 1A, a system of the present invention may, in some embodiments, comprise a single tow band processing line operably connected to a master air jet, wherein the single tow band processing line may be capable of processing at least one continuous tow band. In some embodiments of the present invention, one continuous tow band may be processed along a tow band processing line, wherein the tow band processing line includes at least spreaders and tension rollers and optionally additive application capabilities. The processed tow band from the processing line may then be received by a master air jet and bulked into an acquisition distribution layer. The acquisition distribution layer may then be collected, cut, and/or transported to an AHP manufacturing line. As used herein, the terms “continuous tow band” and “tow band” may be used interchangeably and refer to a collection of continuous (e.g., indefinite or extreme length) filaments, generally having no defined twist, and usually being held together with a crimp and/or tackifier. As used herein, the term “bulking,” “bulked,” and derivatives thereof, refers to increasing caliper without substantial spreading laterally. As used herein, the term “caliper” refers to thickness. As used herein, the term “processed tow bands,” and derivatives thereof, refers to a tow band that has been processed along a tow band processing line.

Referring now to a nonlimiting example illustrated in FIG. 1B, a system of the present invention may, in some embodiments, comprise multiple tow band processing lines operably connected to a master air jet, wherein each tow band processing line may individually be capable of processing one or more tow bands. Shown in FIG. 1B are two tow processing lines each capable of processing to continuous tow bands. Another example involves processing four continuous tow bands along two tow band processing lines (i.e., two continuous tow bands per processing line) each including at least spreaders and tension rollers and optionally additive application capabilities. The processed tow bands produced therefrom may be received by a master air jet in a stacked configuration to produce an acquisition distribution layer having a cross-sectional make-up substantially similar to the composition and relative orientation of the processed tow bands as introduced into the master air jet.

Referring now to another nonlimiting example illustrated in FIG. 1C, some embodiments may involve processing four continuous tow bands along three tow band processing lines (one continuous tow band along each of two tow processing lines and two continuous tow bands along a third tow band processing line). The processed tow bands produced from the three tow band processing lines may be received by a master air jet in a side-by-side configuration to produce an acquisition distribution layer having a cross-sectional make-up substantially similar to the composition and relative orientation of the process tow band as introduced into the master air jet.

It should be noted that in FIGS. 1B and 1C processed tow bands from multiple continuous tow bands are depicted as a single processed tow band from their respective tow band processing line. However, in some embodiments, processing a plurality of continuous tow bands along a single tow band processing line may yield a single processed tow band comprising the plurality of continuous tow bands having been processed or a plurality of processed tow bands comprising one or some subset of the continuous tow bands having been processed. By way of nonlimiting example, a system of the present invention may comprise a tow band processing line capable of receiving at least two continuous tow bands and producing a single processed tow band. By way of another nonlimiting example, a system of the present invention may comprise a tow band processing line capable of receiving at least two continuous tow bands and producing the same number of processed tow bands. By way of yet another nonlimiting example, a system of the present invention may comprise a tow band processing line capable of receiving at least three continuous tow bands and producing two processed tow bands.

In some embodiments, producing an acquisition distribution layer from a continuous tow band(s) may comprise processing at least one continuous tow band to form processed tow bands and forming the processed tow band(s), e.g., by combining the processed tow band(s), into an acquisition distribution layer using a master air jet.

One skilled in the art with the benefit of this disclosure should understand that the desired configuration and composition of an ADL will, at least in part, dictate the design of a system of the present invention. Accordingly, an ADL with a complex configuration and/or composition may require a plurality of tow band processing lines (e.g., six or more).

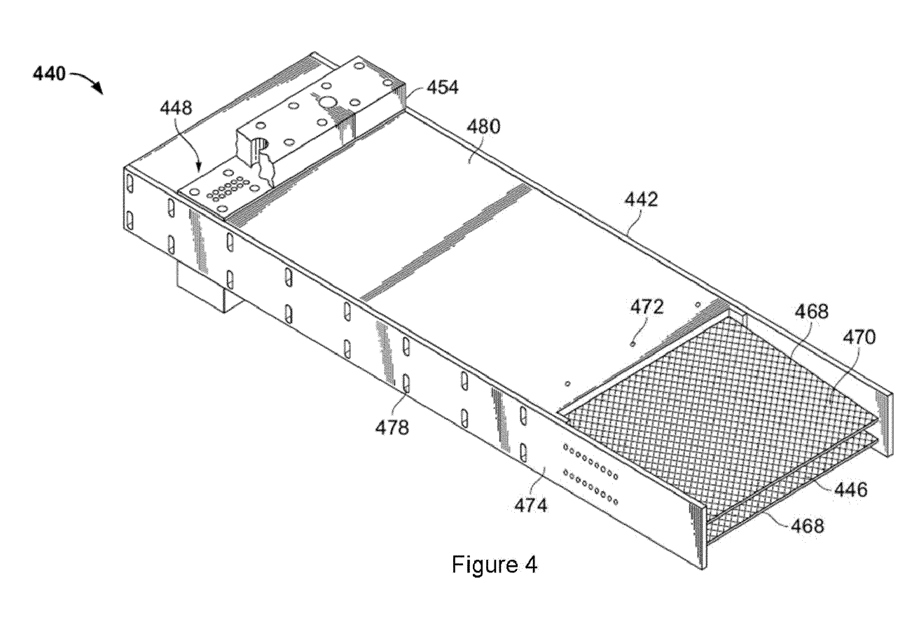

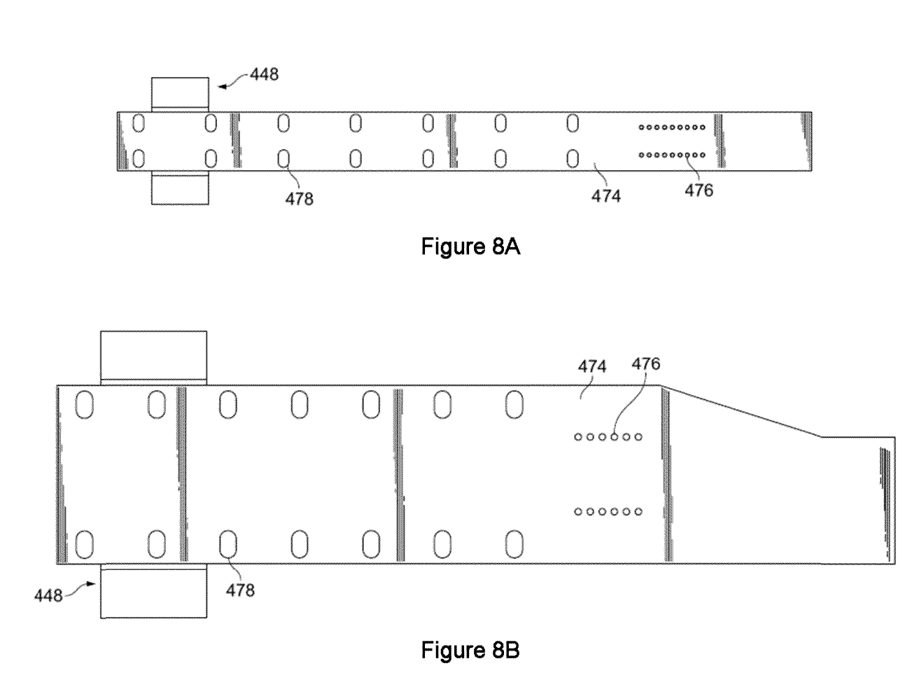

Referring now to FIGS. 4-9, nonlimiting examples of master air jets of the present invention and components thereof, master air jet 440 may include housing 442 that generally is formed by a pair of side plates 474, top plate 480, and bottom plate 482. It should be noted that the terms “side,” “top,” and “bottom” as used to modify the plates are used for simplicity in describing the master air jet and should not be taken to be limiting as to the relation of the master air jet to the plane of the ground. The pair of side plates 474 may be operably attached to the top plate 480 and bottom plate 482 with bolts at sizing guides 478.

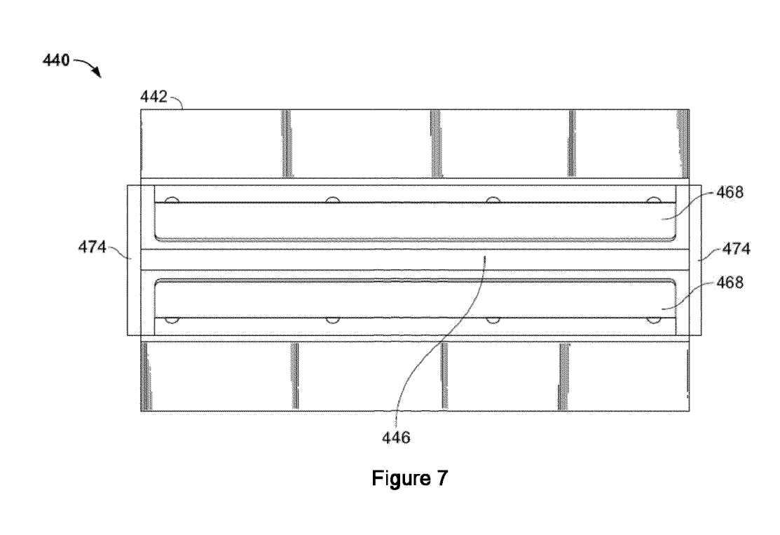

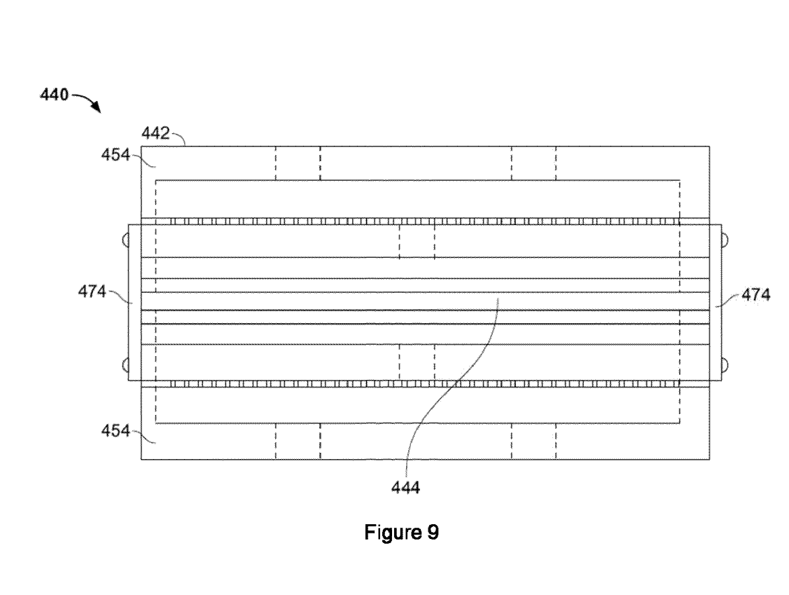

At one end, master air jet 440 includes inlet opening 444. As best seen as an example in FIG. 9, inlet opening 444 may have a generally rectangular configuration that corresponds generally to the shape of the continuous tow band, which is received in inlet opening 444. Housing 442 also includes outlet opening 446 which, as best seen in FIG. 7, may also have a rectangular configuration that corresponds to the desired shape of the processed tow band leaving master air jet 440.

As best seen in FIG. 7, air jet 448 may be formed adjacent the inlet end of housing 442 and may include a source of compressed air (or other fluid in some embodiments) and a conventional control valve for regulating the flow of compressed air from the compressed air source to air manifold 454 through which the compressed air is delivered to jet orifices 456. Jet orifices 456 may form a conventional jet of air for moving the continuous tow band through central passageway 458 in housing 442 as will be explained in greater detail herein. As best seen in FIG. 7, passageway 458 has a gradually increasing cross-sectional area in the direction of movement of the continuous tow band so as to provide forming chamber 460 downstream of air jet 448. Forming chamber 460 may also preferably have a generally rectangular configuration that corresponds to the rectangular shape of the processed tow bands.

Accumulating chamber 462 may be located adjacent the outlet end of housing 442 and downstream of forming chamber 460 and may have a vertical dimension which is greater than outlet opening 446 of forming chamber 460. Accumulating chamber 462 may also be preferably formed with a rectangular configuration to permit the continuous tow band to pass into accumulating chamber 462 from forming chamber 460 to accumulate within accumulating chamber 462. Ultimately the processed tow bands may be withdrawn from housing 442 through outlet opening 446 at different flow rates yielding an acquisition distribution layer.

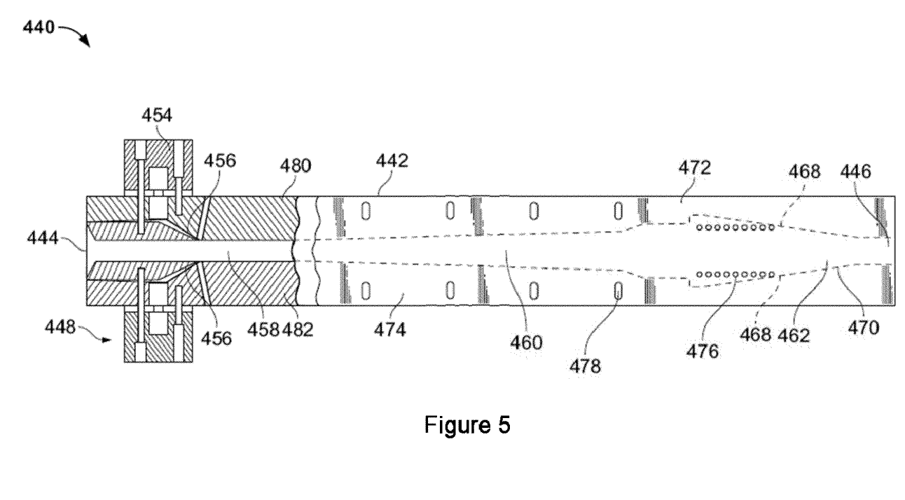

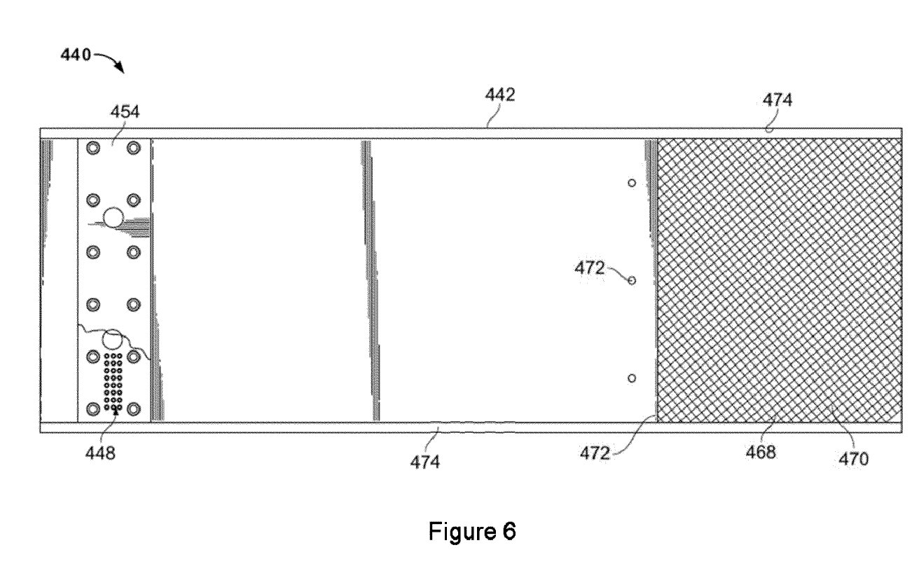

As best seen in FIGS. 5 and 6, a pair of perforated plates 468, each having a large number of perforations 470 therein, may be disposed in accumulating chamber 462 and in side plates 474 between forming chamber 460 and accumulating chamber 462. Perforated plates 468 may be fixed in place to top plate 480 and bottom plate 482 by a plurality of bolts 472 that maintain perforated plates 468 in fixed positions to form accumulating chamber 462.

The relative size of forming chamber 460 and accumulating chamber 462 may help determine the caliper of the acquisition distribution layer produced from master air jet 440. Sizing guides 478 along side plates 474 allow for increasing or decreasing the size of forming chamber 460 to change this effect. It should be noted that the configuration of sizing guides 478 along side pates 474 may allow for changing the size of forming chamber 460 by different amounts by angling top plate 480 relative to bottom plate 482. Varying the shape and/or positions of perforated plates 468 the size of accumulating chamber 462 may be varied.

Similarly, the size of inlet opening 444 and outlet opening 446 may be adjusted using sizing guides 478 along side plates 474 or varying the position and/or shape of perforated plates 468. Variable sizing of inlet opening 444 may advantageously allow for receiving higher caliper processed tow bands into master air jet 440. Also variable sizing of outlet opening 446 may advantageously allow for producing higher caliper an acquisition distribution layer.

As seen best in FIG. 4, side plates 474 may also have a plurality of perforations 476 located generally at a position where the carrier air leaves forming chamber 460 and enters accumulating chamber 462, whereby some of the carrier air can be discharged through perforations 476.

As seen best in FIG. 5, in the operation of master air jet 440, compressed air flows to air jet 448 at a flow rate controlled by the control valve, and it is believed that the jet of air formed by orifices 456 may move the continuous tow band through forming chamber 460. As the processed tow band moves through forming chamber 460 by the carrier air, the carrier air may at least partially bulk the processed tow band so that it gradually increases in cross-sectional area in conformity with the gradually increasing cross-sectional area of forming chamber 460. When the processed tow band exits forming chamber 460 and enters accumulating chamber 462, the processed tow band bulks even further to correspond to the vertical distance between the upstream ends of perforated plates 468 (see FIG. 5).

While some of the carrier air may be discharged through perforations 476 in side plates 474, a substantial portion of the carrier air is believed to move the processed tow band through the spacing between perforated plates 468 and passes outwardly through perforations 470 in perforated plates 468. In so doing, it is believed that the air passing outwardly through perforations 470 urges the processed tow band into frictional engagement with the facing inner surfaces of perforated plates 468. It is believed that this frictional engagement creates a braking action on the processed tow band, which should retard the movement of the processed tow band through accumulating chamber 462. Further, it is believed that the breaking action causes the tow to accumulate in accumulating chamber 462 at a density greater than the processed tow band had in forming chamber 460. Then, the bulked and densified processed tow band exits the accumulating chamber 462 as an acquisition distribution layer through the outlet opening 446 at different flow rates.

It is believed that the flow rate of the carrier air may determine the retarding or braking action applied to the continuous tow band as it passes between perforated plates 468. If the flow rate of the carrier air is increased, the carrier air passing outwardly through perforations 470 in perforated plates 468 will urge the processed tow band into engagement with perforated plates 468 with a greater force, and may thereby increase the retarding or braking action that is applied to the processed tow band. Conversely, if the flow rate of the carrier air is decreased, there will be a smaller braking action applied to the processed tow band. Therefore, a high degree of regulation of the braking action may be obtained by the simple expedient of operating the control valve to provide a flow of carrier air that provides the desired braking action imposed on the processed tow band, which is believed to control the density and caliper of the acquisition distribution layer as it leaves housing 442.

In some embodiments, master air jets of the present invention may having hinged side plates. Hinged side plates may advantageously allow for physically pulling processed tow bands through the master air jet then closing the hinged side plates with the air jets operating so as to create the Venturi that then operates to transport the processed tow bands through the master air jet. By way of nonlimiting example, this pulling action may be desirable when processing high total denier and high denier per filament processed tow bands.

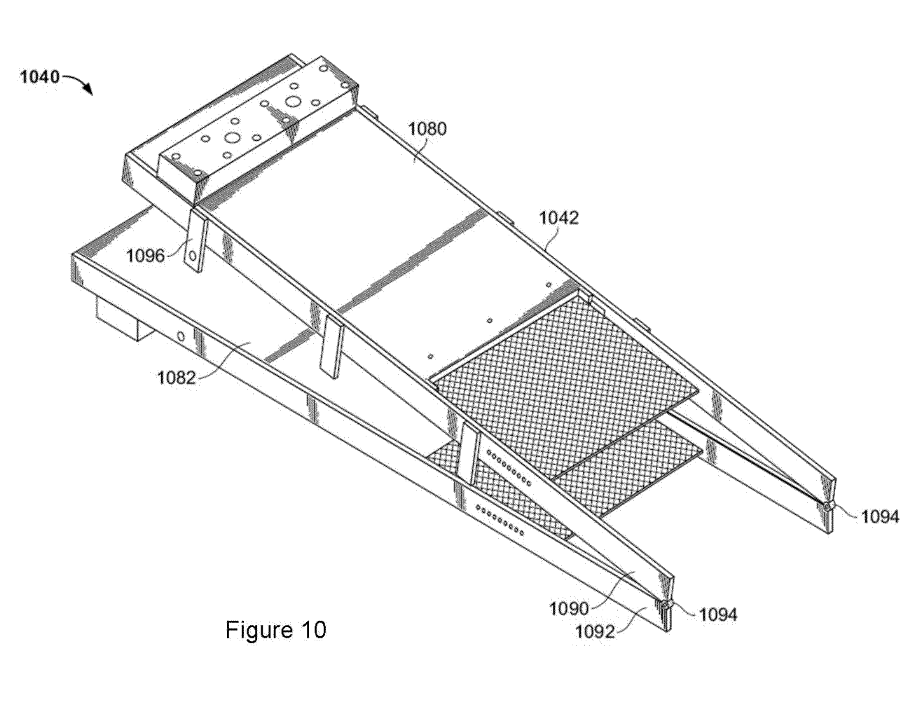

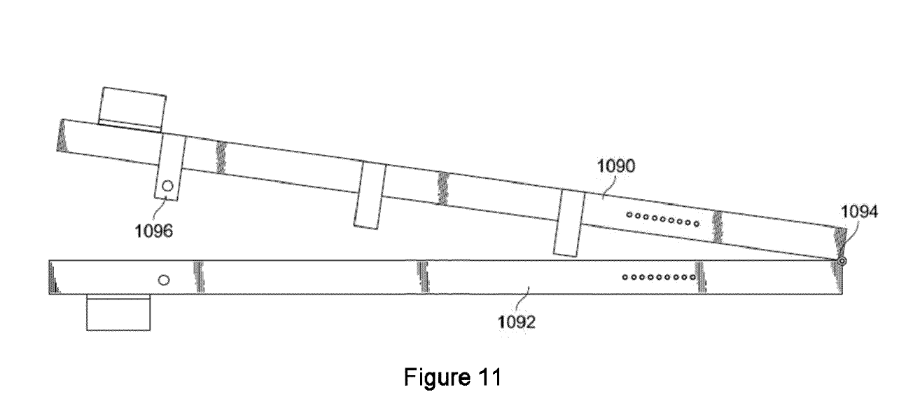

Referring now to FIGS. 10-11, nonlimiting examples of a master air jet of the present invention and components thereof, master air jet 1040 may have a pair of hinged side plates having side plate top half 1090 and side plate bottom half 1092, and side plate hinge 1094. Housing 1042 may be generally formed by top plate 1080 operably attached to side plate top half 1090 and bottom plate 1082 operably attached to side plate bottom half 1092. It should be noted that side, top, and bottom to modify the plates (or components thereof) are used for simplicity in describing the master air jet and should not be taken to be limiting as to the relation of the master air jet to the plane of the ground.

The side plates may have side plate guides 1096 operably attached to either side plate top half 1090 and side plate bottom half 1092 (not shown) to ensure proper alignment when the side plates are closed. To keep the side plate halves 1090 and 1092 closed during operation, at least one side plate guide 1096 may be capable of operably attaching to both side plate halves 1090 and 1092. As shown in FIGS. 10-11, one side plate guide 1096 is attached to side plate top half 1090 and has a hole that lines up with a threaded hole in side plate bottom half 1092 allowing for a bolt to secure side plate halves 1090 and 1092 in the closed position.

One skilled in the art should recognize the plurality of modifications to hinged side plates that achieve the same function of the master air jet, e.g., side plate halves with grooves rather than side plate guides to ensure proper alignment. Further, one skilled in the art should recognize that during operation a processed tow band passing through the master air jet may snag on some imperfections (e.g., burs or gaps) in the side plates, especially at high air jet speeds. Snagging has the potential to adversely affect the edges of the acquisition distribution layer produced and, in some cases, may cause inoperability of the master air jet.

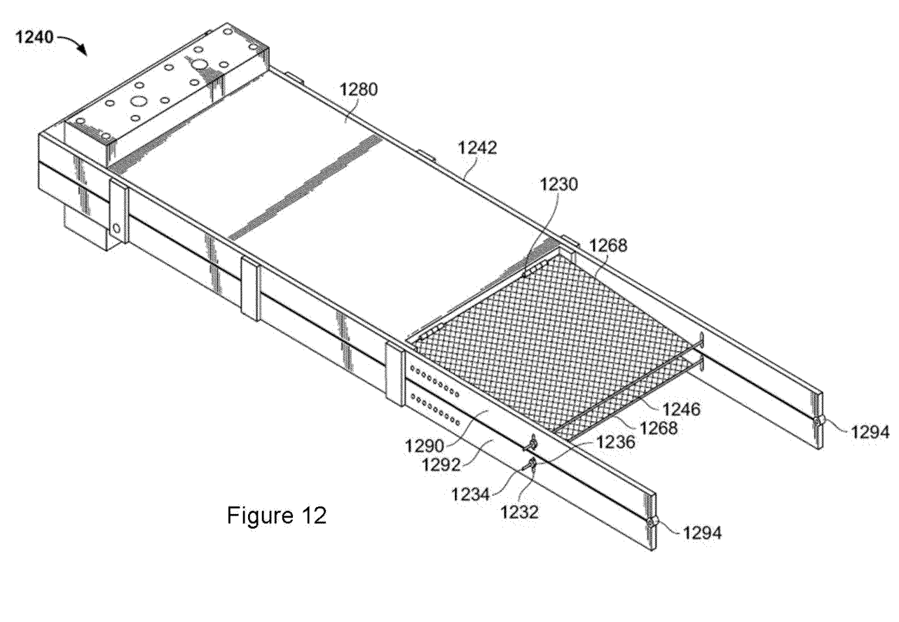

In some embodiments, master air jets of the present invention may have a sizeable outlet opening. Referring now to FIG. 12, a nonlimiting example of a master air jet of the present invention and components thereof, master air jet 1240 may include housing 1242 that generally is formed by a pair of side plates having side plate top half 1290 and side plate bottom half 1292 with side plate hinge 1294; top plate 1280 operably attached to side plate top half 1290, and bottom plate 1282 (not shown) operably attached to side plate bottom half 1292. Accumulating chamber 1262 (not shown) is formed by a pair of perforated plates 1268 fixed in place to top plate 1280 and bottom plate 1282 by hinges 1230 that allow for sizing outlet 1246 by fixing perforated plates 1268 into position by securing perforated plate sizing rods 1234 in outlet sizing guides 1232 with nut 1236.

One skilled in the art should recognize the plurality of modifications to hinged perforated plates that achieve the same function of the master air jet, e.g., vertical screws to adjust the location of the perforated plates and consequently the size of the outlet opening on the fly. One skilled in the art should recognize the modifications should maintain the intended purpose of the perforated plates, i.e., provide a brake for the processed tow bands passing therethrough so as to create the bulk of the subsequent acquisition distribution layer.

In some embodiments, master air jets of the present invention may have any combination of the features including, but not limited to, adjustable side plates, hinged side plates, a sizeable inlet opening, and a sizeable outlet opening. In some embodiments, the present invention provides a master air jet that comprises an inlet opening to a central passageway, the inlet opening having a width of about 5 cm to about 10 m and a height of about 0.5 cm to about 5 cm; an air jet capable of forming a Venturi in a central passageway; a forming chamber along the central passageway disposed after the air jet; an accumulation chamber formed by at least two perforated plates and at least two side plates, the accumulation chamber being disposed along the central passageway after the forming chamber; and an outlet opening to the central passageway, the outlet opening having a width of about 5 cm to about 10 m and a height of about 2 mm to about 500 mm. In some embodiments said master air jet may have a sizeable inlet opening and/or a sizeable outlet opening.

While a master air jet may be configured to have an inlet opening with a width ranging from about 5 cm to about 10 m, a master air jet for use in conjunction with the production of an ADL may preferably have an inlet opening having dimensions of width of about 5 cm to about 15 cm, more preferably about 5 cm to about 10 cm, including any subset therebetween.

While a master air jet may be configured to have an inlet opening with a height ranging from about 0.5 cm to about 5 m, a master air jet for use in conjunction with the production of an ADL may preferably have an inlet opening having dimensions of height of about 0.5 cm to about 3 cm, more preferably about 0.5 cm to about 1 cm, including any subset therebetween.

While a master air jet may be configured to have an outlet opening with a width ranging from about 5 cm to about 10 m, a master air jet for use in conjunction with the production of an ADL may preferably have an outlet opening having dimensions of width of about 5 cm to about 15 cm, more preferably about 5 cm to about 10 cm, including any subset therebetween.

While a master air jet may be configured to have an outlet opening with a height ranging from about 2 mm to about 500 mm, a master air jet for use in conjunction with the production of an ADL may preferably have an outlet opening having dimensions of height of about 2 mm to about 20 mm, more preferably about 2 mm to about 10 mm, including any subset therebetween.

In some embodiments, a system of the present invention may include an additive application area. Suitable additives for use in conjunction with the present invention are detailed further herein. In some embodiments, the system of the present invention may include at least one additive application area. Additive application areas may, in some embodiments, be disposed along at least one tow band processing line, between at least one tow band processing line and a master air jet, and/or after a master air jet. It should be noted that applying includes, but is not limited to, dipping, immersing, submerging, soaking, rinsing, washing, painting, coating, showering, drizzling, spraying, placing, dusting, sprinkling, affixing, and any combination thereof. Further, it should be noted that applying includes, but is not limited to, surface treatments, infusion treatments where the additive incorporates at least partially into filaments, and any combination thereof. One skilled in the art, with the benefit of this disclosure, should understand that an additive should be utilized in a concentration and manner so as to not render the master air jet inoperable for forming an ADL. By way of nonlimiting example, some adhesives and plasticizers should be used in limited quantities as these additives may cause the continuous filaments to stick to a master air jet or not have sufficient mechanical strength to properly form an ADL in the master air jet.

In some embodiments, the application of additives to a tow band (or a processed tow band) may be homogeneous or heterogeneous across the tow band (or a processed tow band). By way of nonlimiting example, along a tow band processing line, an adhesive additive may be applied to the edges of the tow band and an ion exchange resin additive may be applied to the central portion of the tow band. Such a tow band may be used to produce an ADL with adhesive bands at the edges that enhances adhesion to layers above and below the ADL in an AHP and an ion exchange resin additive in the central portion of the ADL that exchanges salt ions and consequently enhances the absorbent capacity of an absorbent core in an AHP. Incorporation of ion exchange resins into an ADL may advantageously exchange salt ions from bodily fluids, which may reduce the level of salt poisoning to the absorbent core and consequently may increase the absorbent capacity of the absorbent core.

Some embodiments of the present invention may involve passing heated gases through the master air jet during formation of the ADL. Some embodiments of the present invention may involve passing inert gases through the master air jet, which may advantageously reduce oxidation of the filament surfaces, especially if the gas is heated. Some embodiments of the present invention may involve passing a heated gas comprising a liquid (e.g., steam) through the master air jet, which may advantageously, for some filament compositions like cellulose acetate, plasticize the filaments and/or increase filament-to-filament bonding.

Some embodiments of the present invention may involve collecting the ADL for storage and/or transporting (e.g., shipping). In some embodiments, a system for producing an ADL of the present invention from tow bands may comprise at least one tow band processing line, at least one master air jet, and a collection area.

Some embodiments of the present invention may involve cutting an ADL into pieces of a desired shape and/or size. In some embodiments, a cut ADL may be continuously produced and fed to an AHP manufacturing line or continuously produced and collected for storage and/or transportation.

In some embodiments, a system of the present invention may be operably connected to an AHP manufacturing line. In some embodiments, an ADL produced from a system of the present invention may be added to an AHP manufacturing line as a continuous ADL. In some embodiments, an ADL produced from a system of the present invention may be cut before addition to an AHP manufacturing line, e.g., a continuous process of production of an ADL using a system of the present invention, cutting the ADL to desired length, and placing the cut ADL on an AHP manufacturing line.

One skilled in the art, with the benefit of this disclosure, will recognize the apparatuses or machinery capable for properly operably connecting (e.g., transporting to, between, and/or from) a tow band processing line, a master air jet, optionally an AHP manufacturing line, or and any additional processing areas or lines (e.g., collection areas, cutting areas, additive application areas, and the like). By way of nonlimiting examples, suitable apparatuses and/or machinery may include guides, rollers, reels, gears, conveyors, transfer belts, vacuums, air jets, and the like, any hybrid thereof, or any combination thereof. By way of nonlimiting example, a system of the present invention may, in some embodiments, include a conveyor for transporting an ADL to a collection area. By way of another nonlimiting example, a system of the present invention may, in some embodiments, include a series of guides and rollers for transporting an ADL from the system directly to an AHP manufacturing line.

### III. Absorbent Hygiene Products

In some embodiments, an ADL of the present invention that comprises continuous filaments may be incorporated as a part of an AHP. Suitable AHP may include, but are not limited to, infant diapers, feminine hygiene pads, adult incontinence products, and the like.

One skilled in the art with the benefit of this disclosure should understand the plurality of AHP configurations in which an ADL of the present invention may be incorporated.

In some embodiments, an AHP may comprise in order: a topsheet, an ADL of the present invention, an absorbent core, and a backsheet. In some embodiments, an AHP may further comprise an adhesive such that the backsheet is disposed between the absorbent core and the adhesive. By way of nonlimiting example, a diaper or incontinence product may comprise in order: a topsheet, an ADL of the present invention, an absorbent core, and a backsheet. By way of another nonlimiting example, a feminine hygiene pad may comprise in order: a topsheet, an ADL of the present invention, an absorbent core, a backsheet, and an adhesive.

In some embodiments, an AHP may comprise an ADL of the present invention disposed about an absorbent core. In some embodiments, an AHP may further comprise a topsheet in a backsheet such that the ADL disposed about the absorbent core is disposed between the topsheet and the backsheet. By way of nonlimiting example, a disposable diaper insert may comprise one surface of the topsheet, an opposing surface of a backsheet, and an ADL of the present invention disposed about the absorbent core that is together disposed between the topsheet and backsheet.

Suitable topsheets for use in conjunction with the present invention may be liquid pervious, permitting liquids to readily penetrate through its thickness, and may be woven materials, nonwoven materials, or films. In some embodiments, for example those that come in contact with the skin, a topsheet for use in conjunction with the present invention may have the characteristics of compliant, soft feeling, and non-irritating to the wearer's skin. Suitable topsheets for use in conjunction with the present invention may comprises materials that include, but are not limited to, porous foams, reticulated foams, apertured plastic films, natural fibers (e.g., wood or cotton fibers), synthetic fibers (e.g., polyester or polypropylene fibers) or from a combination of natural and synthetic fibers. In some embodiments, topsheets for use in conjunction with the present invention may be made of a hydrophobic material to isolate a consumer's skin from liquids in the absorbent core. In some embodiments, topsheets for use in conjunction with the present invention may comprise one or more openings to receive the bodily fluids. In some preferred embodiments, topsheets comprises a compound to adjust hydrophilicity of the material, like a surfactant. In some preferred embodiments, topsheets may comprises a nonwoven material made using means well known to those skilled in the fabrics art.

Preferable topsheets for use in diaper applications may, in some embodiments, have a basis weight from about 10 to about 25 g/m², a minimum dry tensile strength of at least about 150 g/cm in the machine direction, and a strikethrough of less than about 3 seconds according to European Disposables and Nonwovens Association Standard Method 150.4-99. By way of nonlimiting example, one suitable topsheet may comprise a polypropylene spunbonded nonwoven comprises fibers of less than 3 denier per filament having a basis weight of about 18 g/m².

Suitable backsheets for use in conjunction with the present invention may prevent liquids from passing therethrough. For example in diapers and feminine hygiene products, backsheets may prevent fluids absorbed by an absorbent layer and contained within the article from soiling other external articles that may contact the article, such as bed sheets and clothing. Suitable backsheets for use in conjunction with the present invention may include, but are not limited to, thin polymer films of hydrophobic polymers like polypropylene and polyethylene. By way of nonlimiting example, a backsheet for use in conjunction with the present invention may comprise a layer of polyethylene film having a basis weight between about 10 g/m² and about 30 g/m², although other flexible, liquid impervious materials can be used. In some embodiments, backsheets for use in conjunction with the present invention are breathable (e.g., via micropores) so as to permit vapors to escape from the diaper while still preventing fluids (e.g., exudates) from passing therethrough. In some embodiments, backsheets for use in conjunction with the present invention may have a nonwoven laminated to the film layer so as to make backsheet more “cloth-like”. Such a nonwoven layer may, in some embodiments, comprise a nonwoven material (e.g., one having a spunbonded or other suitable structure) with a basis weight between about 15 g/m² and about 25 g/m².

Suitable adhesives for use in conjunction with the present invention may include, but are not limited to, pressure sensitive adhesives, glue, gelatin, caesin, starch, cellulose esters, aliphatic polyesters, poly(alkanoates), aliphatic-aromatic polyesters, sulfonated aliphatic-aromatic polyesters, polyamide esters, rosin/polycaprolactone triblock copolymers, rosin/poly(ethylene adipate) triblock copolymers, rosin/poly(ethylene succinate) triblock copolymers, poly(vinyl acetates), poly(ethylene-co-ethylacrylate), poly(ethylene-co-methyl acrylate), poly(ethylene-co-propylene), poly(ethylene-co-1-butene), poly(ethylene-co-1-pentene), poly(styrene), acrylics, polyurethanes, sulfonated polyester urethane dispersions, nonsulfonated urethane dispersions, urethane-styrene polymer dispersions, non-ionic polyester urethane dispersions, acrylic dispersions, silyl anionic acrylate-styrene polymer dispersions, anionic acrylate-styrene dispersions, anionic acrylate-styrene-acrylonitrile dispersions, acrylate-acrylonitrile dispersions, vinylchloride-ethylene emulsions, vinylpyrrolidone/styrene copolymer emulsions, carboxylated and noncarboxylated vinyl acetate ethylene dispersions, vinyl acetate homopolymer dispersions, polyvinyl chloride emulsions, polyvinylidene fluoride dispersions, ethylene acrylic acid dispersions, polyamide dispersions, anionic carboxylated or noncarboxylated acrylonitrile-butadiene-styrene emulsions and acrylonitrile emulsions, resin dispersions derived from styrene, resin dispersions derived from aliphatic and/or aromatic hydrocarbons, styrene-maleic anhydrides, gamma-chloropropylmethoxysilane, vinyltrichlorosilane, vinyltriethoxysilane, vinyltris(beta-methoxyethoxy)silane, gamma-methacryloxypropyltrimethoxysilane, beta-(3,4-epoxycyclohexyl)ethyltrimethoxysilane, gammaglycidoxypropyltrimethoxysilane, vinyl-triacetoxysilane, gamma-mercaptopropyltrimethoxysilane, gamma-aminopropyltriethoxysilane, N-beta-(aminoethyl)-gamma-aminopropyl-trimethoxysilane, and the like, or any combination thereof. Said adhesives may be applied through melt processes or through solution, emulsion, dispersion, or other suitable coating processes.

### IV. Optional Additives

Suitable additives for optional use in conjunction with the present invention may include, but not be limited to, active particles, active compounds, ion exchange resins, superabsorbent polymers, zeolites, nanoparticles, ceramic particles, abrasive particulates, absorbent particulates, softening agents, plasticizers, pigments, dyes, aromas, controlled release vesicles, binders, adhesives, tackifiers, surface modification agents, lubricating agents, emulsifiers, vitamins, peroxides, biocides, antifungals, antimicrobials, deodorizers, antistatic agents, flame retardants, antifoaming agents, degradation agents, conductivity modifying agents, stabilizing agents, or any combination thereof. Said additives are detailed further herein.

Active particles for use in conjunction with the present invention may be useful in actively reducing components from a fluid stream by absorption or reaction. Suitable active particles for use in conjunction with the present invention may include, but not be limited to, nano-scaled carbon particles, carbon nanotubes having at least one wall, carbon nanohorns, bamboo-like carbon nanostructures, fullerenes, fullerene aggregates, graphene, few layer graphene, oxidized graphene, iron oxide nanoparticles, nanoparticles, metal nanoparticles, gold nanoparticles, silver nanoparticles, metal oxide nanoparticles, alumina nanoparticles, magnetic nanoparticles, paramagnetic nanoparticles, superparamagnetic nanoparticles, gadolinium oxide nanoparticles, hematite nanoparticles, magnetite nanoparticles, gado-nanotubes, endofullerenes, Gd@C₆₀, core-shell nanoparticles, onionated nanoparticles, nanoshells, onionated iron oxide nanoparticles, activated carbon, ion exchange resins, desiccants, silicates, molecular sieves, silica gels, activated alumina, zeolites, perlite, sepiolite, Fuller's Earth, magnesium silicate, metal oxides, iron oxides, activated carbon, and any combination thereof.

Suitable active particles for use in conjunction with the present invention may have at least one dimension of about less than one nanometer, such as graphene, to as large as a particle having a diameter of about 5000 nanometers. Active particles for use in conjunction with the present invention may range from a lower size limit in at least one dimension of about: 0.1 nanometers, 0.5 nanometers, 1 nanometer, 10 nanometers, 100 nanometers, 500 nanometers, 1 micron, 5 microns, 10 microns, 50 microns, 100 microns, 150 microns, 200 microns, and 250 microns. The active particles may range from an upper size limit in at least one dimension of about: 5000 microns, 2000 microns, 1000 microns, 900 microns, 700 microns, 500 microns, 400 microns, 300 microns, 250 microns, 200 microns, 150 microns, 100 microns, 50 microns, 10 microns, and 500 nanometers. Any combination of lower limits and upper limits above may be suitable for use in conjunction with the present invention, wherein the selected maximum size is greater than the selected minimum size. In some embodiments, the active particles for use in conjunction with the present invention may be a mixture of particle sizes ranging from the above lower and upper limits. In some embodiments of the present invention, the size of the active particles may be polymodal.

Active compounds for use in conjunction with the present invention may be useful in actively reducing components from a fluid stream by absorption or reaction. Suitable active compounds for use in conjunction with the present invention may include, but not be limited to, malic acid, potassium carbonate, citric acid, tartaric acid, lactic acid, ascorbic acid, polyethyleneimine, cyclodextrin, sodium hydroxide, sulphamic acid, sodium sulphamate, polyvinyl acetate, carboxylated acrylate, or any combination thereof.

Suitable ion exchange resins for use in conjunction with the present invention may include, but not be limited to, polymers with a backbone, such as styrene-divinyl benezene (DVB) copolymer, acrylates, methacrylates, phenol formaldehyde condensates, and epichlorohydrin amine condensates; a plurality of electrically charged functional groups attached to the polymer backbone; or any combination thereof.

As used herein, the term “superabsorbent materials” refers to materials, e.g., polymers, capable of absorbing at least three times their weight of a fluid. Suitable superabsorbent materials for use in conjunction with the present invention may include, but not be limited to, sodium polyacrylate, starch graved copolymers of polyacrylonitriles, polyvinyl alcohol copolymers, cross-linked poly(ethylene oxides), polyacrylamide copolymers, ethylene maleic anhydride copolymers, cross-linked carboxymethylcelluloses, and the like, or any combination thereof. By way of nonlimiting example, superabsorbent materials incorporated into a nonwoven may be useful in chemical spill rags and kits.

Zeolites for use in conjunction with the present invention may include crystalline aluminosilicates having pores, e.g., channels, or cavities of uniform, molecular-sized dimensions. Zeolites may include natural and synthetic materials. Suitable zeolites may include, but not be limited to, zeolite BETA (Na₇(Al₇Si₅₇O₁₂₈) tetragonal), zeolite ZSM-5 (Naₙ(AlₙSi₉₆₋ₙO₁₉₂) 16H₂O, with n<27), zeolite A, zeolite X, zeolite Y, zeolite K-G, zeolite ZK-5, zeolite ZK-4, mesoporous silicates, SBA-15, MCM-41, MCM48 modified by 3-aminopropylsilyl groups, alumino-phosphates, mesoporous aluminosilicates, other related porous materials (e.g., such as mixed oxide gels), or any combination thereof.

Suitable nanoparticles for use in conjunction with the present invention may include, but not be limited to, nano-scaled carbon particles like carbon nanotubes of any number of walls, carbon nanohorns, bamboo-like carbon nanostructures, fullerenes and fullerene aggregates, and graphene including few layer graphene and oxidized graphene; metal nanoparticles like gold and silver; metal oxide nanoparticles like alumina, silica, and titania; magnetic, paramagnetic, and superparamagentic nanoparticles like gadolinium oxide, various crystal structures of iron oxide like hematite and magnetite, about 12 nm Fe₃O₄, gado-nanotubes, and endofullerenes like Gd@C₆₀; and core-shell and onionated nanoparticles like gold and silver nanoshells, onionated iron oxide, and others nanoparticles or microparticles with an outer shell of any of said materials; and any combination of the foregoing. It should be noted that nanoparticles may include nanorods, nanospheres, nanorices, nanowires, nanostars (like nanotripods and nanotetrapods), hollow nanostructures, hybrid nanostructures that are two or more nanoparticles connected as one, and non-nano particles with nano-coatings or nano-thick walls. It should be further noted that nanoparticles for use in conjunction with the present invention may include the functionalized derivatives of nanoparticles including, but not limited to, nanoparticles that have been functionalized covalently and/or non-covalently, e.g., pi-stacking, physisorption, ionic association, van der Waals association, and the like. Suitable functional groups may include, but not be limited to, moieties comprising amines (1°, 2°, or 3°), amides, carboxylic acids, aldehydes, ketones, ethers, esters, peroxides, silyls, organosilanes, hydrocarbons, aromatic hydrocarbons, and any combination thereof; polymers; chelating agents like ethylenediamine tetraacetate, diethylenetriaminepentaacetic acid, triglycollamic acid, and a structure comprising a pyrrole ring; and any combination thereof.

Suitable ceramic particles for use in conjunction with the present invention may include, but not be limited to, oxides (e.g., silica, titania, alumina, beryllia, ceria, and zirconia), nonoxides (e.g., carbides, borides, nitrides, and silicides), composites thereof, or any combination thereof. Ceramic particles may be crystalline, non-crystalline, or semi-crystalline.

Suitable softening agents and/or plasticizers for use in conjunction with the present invention may include, but not be limited to, water, glycerol triacetate (triacetin), triethyl citrate, dimethoxy-ethyl phthalate, dimethyl phthalate, diethyl phthalate, methyl phthalyl ethyl glycolate, o-phenyl phenyl-(bis) phenyl phosphate, 1,4-butanediol diacetate, diacetate, dipropionate ester of triethylene glycol, dibutyrate ester of triethylene glycol, dimethoxyethyl phthalate, triethyl citrate, triacetyl glycerin, and the like, any derivative thereof, and any combination thereof. One skilled in the art with the benefit of this disclosure should understand the concentration of plasticizers to use as an additive to the filaments.

As used herein, pigments refer to compounds and/or particles that impart color and are incorporated throughout the filaments. Suitable pigments for use in conjunction with the present invention may include, but not be limited to, titanium dioxide, silicon dioxide, carbon black, tartrazine, E102, phthalocyanine blue, phthalocyanine green, quinacridones, perylene tetracarboxylic acid di-imides, dioxazines, perinones disazo pigments, anthraquinone pigments, carbon black, metal powders, iron oxide, ultramarine, calcium carbonate, kaolin clay, aluminum hydroxide, barium sulfate, zinc oxide, aluminum oxide, caramel, fruit or vegetable or spice colorants (e.g., beet powder, beta-carotene, turmeric, paprika), or any combination thereof.

As used herein, dyes refer to compounds and/or particles that impart color and are a surface treatment of the filaments. Suitable dyes for use in conjunction with the present invention may include, but not be limited to, CARTASOL® dyes (cationic dyes, available from Clariant Services) in liquid and/or granular form (e.g., CARTASOL® Brilliant Yellow K-6G liquid, CARTASOL® Yellow K-4GL liquid, CARTASOL® Yellow K-GL liquid, CARTASOL® Orange K-3GL liquid, CARTASOL® Scarlet K-2GL liquid, CARTASOL® Red K-3BN liquid, CARTASOL® Blue K-5R liquid, CARTASOL® Blue K-RL liquid, CARTASOL® Turquoise K-RL liquid/granules, CARTASOL® Brown K-BL liquid), FASTUSOL® dyes (an auxochrome, available from BASF) (e.g., Yellow 3GL, Fastusol C Blue 74L).

Suitable aromas for use in conjunction with the present invention may include, but not be limited to, methyl formate, methyl acetate, methyl butyrate, ethyl acetate, ethyl butyrate, isoamyl acetate, pentyl butyrate, pentyl pentanoate, octyl acetate, myrcene, geraniol, nerol, citral, citronellal, citronellol, linalool, nerolidol, limonene, camphor, terpineol, alpha-ionone, thujone, benzaldehyde, eugenol, cinnamaldehyde, ethyl maltol, vanilla, anisole, anethole, estragole, thymol, furaneol, methanol, or any combination thereof.

Suitable binders for use in conjunction with the present invention may include, but not be limited to, polyolefins, polyesters, polyamides (or nylons), polyacrylics, polystyrenes, polyvinyls, polytetrafluoroethylene (PTFE), polyether ether ketone (PEEK), any copolymer thereof, any derivative thereof, and any combination thereof. Non-fibrous plasticized cellulose derivatives may also be suitable for use as binder particles in the present invention. Examples of suitable polyolefins may include, but not be limited to, polyethylene, polypropylene, polybutylene, polymethylpentene, and the like, any copolymer thereof, any derivative thereof, and any combination thereof. Examples of suitable polyethylenes may include, but not be limited to, ultrahigh molecular weight polyethylene, very high molecular weight polyethylene, high molecular weight polyethylene, low-density polyethylene, linear low-density polyethylene, high-density polyethylene, and the like, any copolymer thereof, any derivative thereof, and any combination thereof. Examples of suitable polyesters may include, but not be limited to, polyethylene terephthalate, polybutylene terephthalate, polycyclohexylene dimethylene terephthalate, polytrimethylene terephthalate, and the like, any copolymer thereof, any derivative thereof, and any combination thereof. Examples of suitable polyacrylics may include, but not be limited to, polymethyl methacrylate, and the like, any copolymer thereof, any derivative thereof, and any combination thereof. Examples of suitable polystyrenes may include, but not be limited to, polystyrene, acrylonitrile-butadiene-styrene, styrene-acrylonitrile, styrene-butadiene, styrene-maleic anhydride, and the like, any copolymer thereof, any derivative thereof, and any combination thereof. Examples of suitable polyvinyls may include, but not be limited to, ethylene vinyl acetate, ethylene vinyl alcohol, polyvinyl chloride, and the like, any copolymer thereof, any derivative thereof, and any combination thereof. Examples of suitable cellulosics may include, but not be limited to, cellulose acetate, cellulose acetate butyrate, plasticized cellulosics, cellulose propionate, ethyl cellulose, and the like, any copolymer thereof, any derivative thereof, and any combination thereof. In some embodiments, binder particles may comprise any copolymer, any derivative, or any combination of the above listed binders. Further, binder particles may be impregnated with and/or coated with any combination of additives disclosed herein.

Suitable tackifiers for use in conjunction with the present invention may include, but not be limited to, methylcellulose, ethylcellulose, hydroxyethylcellulose, carboxy methylcellulose, carboxy ethylcellulose, water-soluble cellulose acetate, amides, diamines, polyesters, polycarbonates, silyl-modified polyamide compounds, polycarbamates, urethanes, natural resins, shellacs, acrylic acid polymers, 2-ethylhexylacrylate, acrylic acid ester polymers, acrylic acid derivative polymers, acrylic acid homopolymers, anacrylic acid ester homopolymers, poly(methyl acrylate), poly(butyl acrylate), poly(2-ethylhexyl acrylate), acrylic acid ester co-polymers, methacrylic acid derivative polymers, methacrylic acid homopolymers, methacrylic acid ester homopolymers, poly(methyl methacrylate), poly(butyl methacrylate), poly(2-ethylhexyl methacrylate), acrylamido-methyl-propane sulfonate polymers, acrylamido-methyl-propane sulfonate derivative polymers, acrylamido-methyl-propane sulfonate co-polymers, acrylic acid/acrylamido-methyl-propane sulfonate co-polymers, benzyl coco di-(hydroxyethyl) quaternary amines, p-T-amyl-phenols condensed with formaldehyde, dialkyl amino alkyl(meth)acrylates, acrylamides, N-(dialkyl amino alkyl) acrylamide, methacrylamides, hydroxy alkyl(meth)acrylates, methacrylic acids, acrylic acids, hydroxyethyl acrylates, and the like, any derivative thereof, or any combination thereof.

Suitable lubricating agents for use in conjunction with the present invention may include, but not be limited to, ethoxylated fatty acids (e.g., the reaction product of ethylene oxide with pelargonic acid to form poly(ethylene glycol) (“PEG”) monopelargonate; the reaction product of ethylene oxide with coconut fatty acids to form PEG monolaurate), and the like, or any combination thereof. The lubricant agents may also be selected from nonwater-soluble materials such as synthetic hydrocarbon oils, alkyl esters (e.g., tridecyl stearate which is the reaction product of tridecyl alcohol and stearic acid), polyol esters (e.g., trimethylol propane tripelargonate and pentaerythritol tetrapelargonate), and the like, or any combination thereof.

Suitable emulsifiers for use in conjunction with the present invention may include, but not be limited to, sorbitan monolaurate, e.g., SPAN® 20 (available from Uniqema, Wilmington, Del.), or poly(ethylene oxide) sorbitan monolaurate, e.g., TWEEN® 20 (available from Uniqema, Wilmington, Del.).

Suitable vitamins for use in conjunction with the present invention may include, but not be limited to, vitamin B compounds (including B1 compounds, B2 compounds, B3 compounds such as niacinamide, niacinnicotinic acid, tocopheryl nicotinate, C₁-C₁₈ nicotinic acid esters, and nicotinyl alcohol; B5 compounds, such as panthenol or “pro-B5”, pantothenic acid, pantothenyl; B6 compounds, such as pyroxidine, pyridoxal, pyridoxamine; carnitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids; or any combination thereof.

Suitable antimicrobials for use in conjunction with the present invention may include, but not be limited to, anti-microbial metal ions, chlorhexidine, chlorhexidine salt, triclosan, polymoxin, tetracycline, amino glycoside (e.g., gentamicin), rifampicin, bacitracin, erythromycin, neomycin, chloramphenicol, miconazole, quinolone, penicillin, nonoxynol 9, fusidic acid, cephalosporin, mupirocin, metronidazolea secropin, protegrin, bacteriolcin, defensin, nitrofurazone, mafenide, acyclovir, vanocmycin, clindamycin, lincomycin, sulfonamide, norfloxacin, pefloxacin, nalidizic acid, oxalic acid, enoxacin acid, ciprofloxacin, polyhexamethylene biguanide (PHMB), PHMB derivatives (e.g., biodegradable biguanides like polyethylene hexamethylene biguanide (PEHMB)), clilorhexidine gluconate, chlorohexidine hydrochloride, ethylenediaminetetraacetic acid (EDTA), EDTA derivatives (e.g., disodium EDTA or tetrasodium EDTA), and the like, and any combination thereof.

Antistatic agents (antistats) for use in conjunction with the present invention may comprise any suitable anionic, cationic, amphoteric or nonionic antistatic agent. Anionic antistatic agents may generally include, but not be limited to, alkali sulfates, alkali phosphates, phosphate esters of alcohols, phosphate esters of ethoxylated alcohols, or any combination thereof. Examples may include, but not be limited to, alkali neutralized phosphate ester (e.g., TRYFAC® 5559 or TRYFRAC® 5576, available from Henkel Corporation, Mauldin, S.C.). Cationic antistatic agents may generally include, but not be limited to, quaternary ammonium salts and imidazolines which possess a positive charge. Examples of nonionics include the poly(oxyalkylene) derivatives, e.g., ethoxylated fatty acids like EMEREST® 2650 (an ethoxylated fatty acid, available from Henkel Corporation, Mauldin, S.C.), ethoxylated fatty alcohols like TRYCOL® 5964 (an ethoxylated lauryl alcohol, available from Henkel Corporation, Mauldin, S.C.), ethoxylated fatty amines like TRYMEEN® 6606 (an ethoxylated tallow amine, available from Henkel Corporation, Mauldin, S.C.), alkanolamides like EMID® 6545 (an oleic diethanolamine, available from Henkel Corporation, Mauldin, S.C.), or any combination thereof. Anionic and cationic materials tend to be more effective antistats.

To facilitate a better understanding of the present invention, the following examples of preferred embodiments are given. In no way should the following examples be read to limit, or to define, the scope of the invention.

### EXAMPLES

### Example 1

To combine two tow bands, a system was used that included a tow band processing line having three tow spreaders, a master air jet for receiving the tow bands from the tow band processing line, hand-held guidery between the first and second tow spreaders of the tow band processing line, and hand-held guidery between the tow band processing line and master air jet. Two tow bands were spread on the tow band processing lines then introduced in a stacked configuration into the master air jet. One tow band was colored while the other tow band was white. The produced ADL produced has intermingling at the interface of the two tow bands and has sidedness with one side being substantially the colored tow band and the other side being substantially the white tow band. This example demonstrates the cross-sectional make-up of the ADL is substantially the same as the composition and positional relationship of the processed tow bands as introduced into the master air jet.

### Example 2

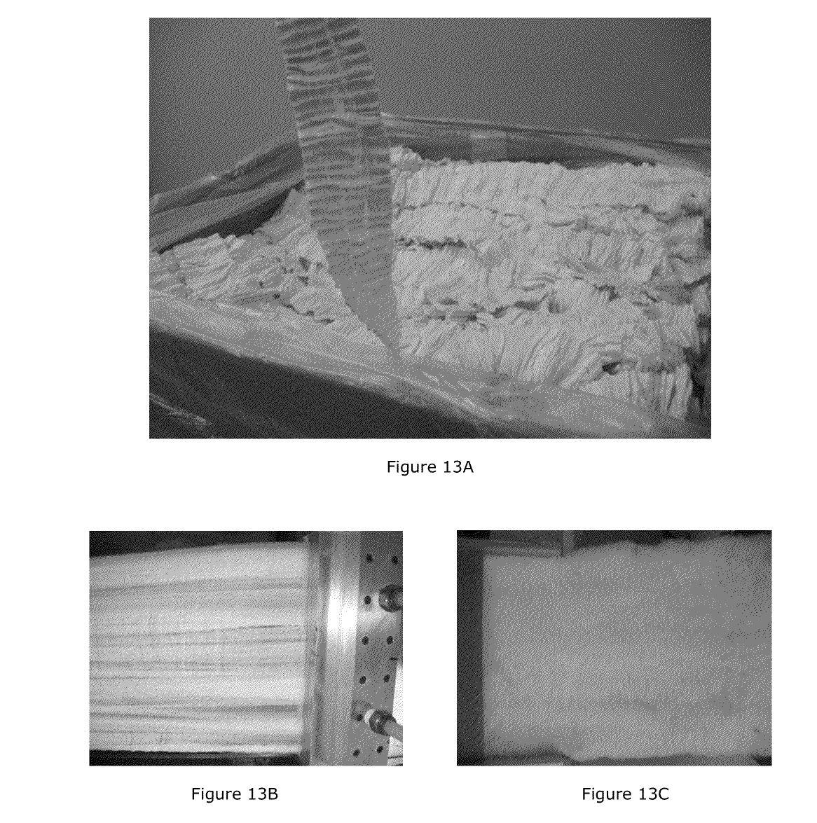

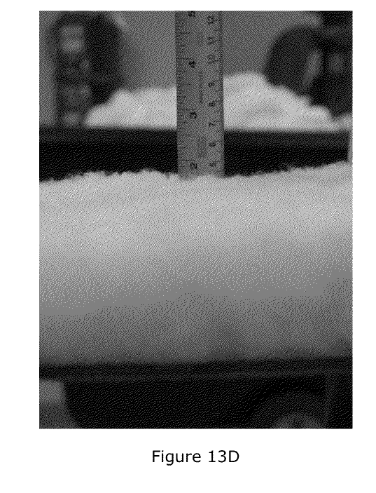

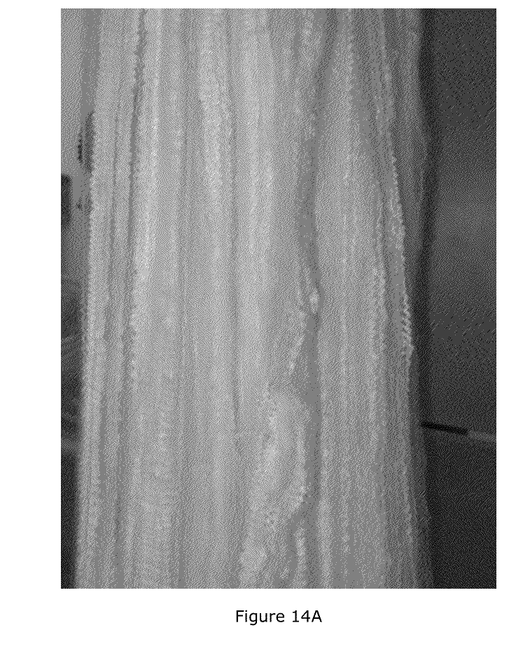

A polyester tow band having 280,000 total denier, 2.25 dpf, 44 crimps/10 cm, and a 4 inch width, shown in FIG. 13A was run along a tow band processing line having 3 spreaders and a delivery roll and into a master air jet. The master air jet had an inlet width of 250 mm, air pressure of 60 psig, inlet height of 10 mm, and outlet height of 39 mm. The bulked polyester tow band as introduced into the master air jet had substantially the same width, approximately 10 inches, as the ADL exiting the master air jet, as shown in FIGS. 13B, and 13C, respectively. The produced ADL had a caliper of about 4.5 cm as shown in FIG. 13D. It should be noted that the caliper of the resultant consolidated web was greater than the outlet height of the master air jet.

While an ADL may more preferably be a smaller caliper, this example demonstrates the efficacy of a system according to at least one embodiment of the present invention in producing bulked ADL-like materials from continuous tow bands.

### Example 3

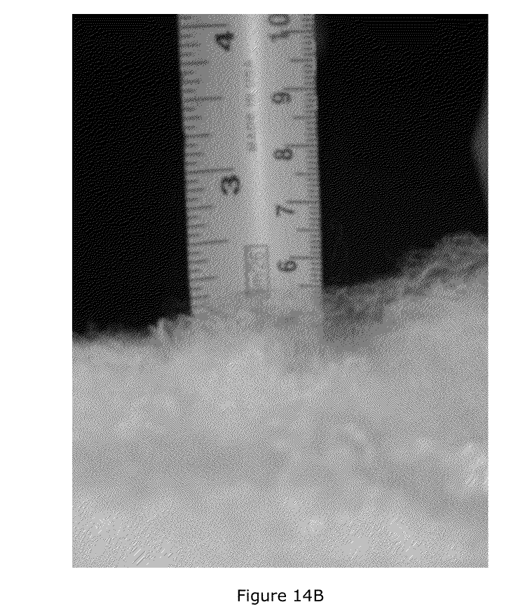

A section of 3,000,000 tow band was extracted yielding about a 200,000 total denier tow band. A lyocell tow band having a 24-inch substantially circular cross-section (as compared to the rectangular cross-section of Example 2) with a total denier of about 350,000, 3 dpf filaments, and 30 crimps/10 cm, shown after spreading in FIG. 14A, was processed through the same procedure as Example 2 to produce an ADL having a caliper of about 5.5 cm as shown in FIG. 14B. It should be noted that similar to Example 2 the caliper of the resultant consolidated web was greater than the outlet height of the master air jet.

While an ADL may more preferably be a smaller caliper, this example demonstrates the efficacy of a system according to at least one embodiment of the present invention in producing bulked ADL-like materials from continuous tow bands.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

The invention claimed is:

1. A system comprising:
a tow band processing line; and
a master air jet in operably connected to the tow band processing line to receive a processed tow band from the tow band processing line so as to produce an acquisition distribution layer for an absorbent article.

2. The system of claim 1 further comprising:
an absorbent hygiene product manufacturing line operably connected to the master air jet to receive the acquisition distribution layer.

3. The system of claim 2 further comprising:
a packaging area disposed after the absorbent hygiene product manufacturing line.

4. The system of claim 1 further comprising:
at least one additive area disposed at a location selected from the group consisting of along the tow band processing line, between the tow band processing line and the master air jet, and after the master air jet.

5. A method comprising:
producing an acquisition distribution layer from a plurality of processed tow bands using a master air jet; and
forming an absorbent hygiene product from the acquisition distribution layer.

6. The method of claim 5, wherein the plurality of processed tow bands are positionally stacked, side-by-side, or a combination thereof.

7. The method of claim 5, wherein the acquisition distribution layer has a bulk density of about 0.05 g/cm³ or less.

8. The method of claim 5, wherein the acquisition distribution layer has a caliper of about 2 mm to about 10 mm.

9. The method of claim 5, wherein the acquisition distribution layer has a width of about 1 cm to about 10 cm.

10. An absorbent hygiene product produced by the method of claim 5.

11. An acquisition distribution layer comprising:
a plurality of continuous filaments bulked by a master air jet.

12. The acquisition distribution layer of claim 11, wherein the plurality of continuous filaments comprise at least two different filament compositions.

13. The acquisition distribution layer of claim 11, wherein the acquisition distribution layer has a heterogeneous cross-sectional make-up.

14. The acquisition distribution layer of claim 11, wherein the acquisition distribution layer has a layered cross-sectional make-up.

15. The acquisition distribution layer of claim 11, wherein the acquisition distribution layer has a bulk density of about 0.05 g/cm³ or less.

16. The acquisition distribution layer of claim 11, wherein the acquisition distribution layer has a caliper of about 0.1 mm to about 10 mm.

17. The acquisition distribution layer of claim 11, wherein the acquisition distribution layer has a width of about 1 cm to about 10 cm.

18. An absorbent hygiene product comprising, in order:
a topsheet;
an acquisition distribution layer comprising a plurality of continuous filaments bulked by a master air jet;
an absorbent core; and
a backsheet.

19. The absorbent hygiene product of claim 18 further comprising:
an adhesive layer such that the backsheet is disposed between the absorbent core and the adhesive layer.
